(19) United States
(12) Patent Application Publication
(10) Pub. No.: US 2024/0252582 A1
(43) Pub. Date: Aug. 1, 2024

(54) METHODS FOR TREATING AMBLYOPIA

(71) Applicant: City University of Hong Kong, Kowloon (HK)

(72) Inventors: Jufang He, Kowloon (HK); Leanne Lai Hang Chan, Kowloon (HK); Anju Malik, Kowloon (HK); Xuejiao Zheng, Shatin (HK)

(21) Appl. No.: 18/162,927

(22) Filed: Feb. 1, 2023

(51) Int. Cl. A61K 38/08; A61K 31/506; A61K 38/07; A61P 27/10

(52) U.S. Cl. CPC A61K 38/08; A61K 31/506; A61K 38/07; A61P 27/10

(57) ABSTRACT

A method for treating or preventing amblyopia includes administrating a therapeutically effective amount of cholecystokinin receptor B (CCKBR) agonist in a subject in need thereof. A method of regaining or improving visual acuity includes administrating a therapeutically effective amount of CCKBR agonist in a subject in need thereof. A CCKBR agonist is used in the manufacture of a medicament for treating or preventing amblyopia in a subject in need thereof.

### FIELD OF THE INVENTION

The present invention relates to a method for treating amblyopia.

### BACKGROUND

Amblyopia is decreased vision in one or both eyes due to abnormal vision development in early life. In amblyopia, vision loss occurs because nerve pathways between the brain and the eye are not properly stimulated in the first few years of life (American Association for Pediatric Ophthalmology & Strabismus, https://aapos.org/glossary/amblyopia).

Current options for treating amblyopia include occlusion therapy (such as wearing an eye patch on the normal eye to excise the weaker eye), optical approaches (such as wearing corrective lenses and glasses), and pharmaceutical agents (such as Levodopa and Carbidopa). However, all of these options suffer from certain drawbacks. For example, eye patches need to be used for long periods and patients usually show poor adherence to such therapy. Optical approaches are usually only applicable for amblyopia resulting from nearsightedness, farsightedness, astigmatism, etc. Levodopa's taste is unpleasant and causes side effects. Carbidopa cannot cross the blood-brain barrier and only prevents levodopa conversion peripherally while allowing more central levodopa activity. Furthermore, adults' results from traditional amblyopia therapies are generally deficient because, without proper excises at early age, nerve pathways between the brain and the weaker eye cannot be restored easily.

Accordingly, there is a great need in the art for new therapies for treating and/or preventing amblyopia, while avoiding the shortcomings and draw backs of prior art treatments.

### SUMMARY OF THE INVENTION

An embodiment of the present invention relates to a method for treating or preventing amblyopia by administrating a therapeutically effective amount of cholecystokinin receptor B (CCKBR) agonist in a subject in need thereof.

An embodiment of the present invention relates to a method of regaining or improving visual acuity by administrating a therapeutically effective amount of CCKBR agonist in a subject in need thereof.

An embodiment of the present invention relates to use of a CCKBR agonist in the manufacture of a medicament for treating or preventing amblyopia in a subject in need thereof.

Without intending to be limited by theory it is believed that the present invention may provide a new therapy for treating and/or preventing amblyopia, which can facilitate the recovery of lost visual plasticity, especially in adults.

### BRIEF DESCRIPTION OF THE DRAWINGS

FIGS. 1A and 1B show embodiments of the experimental design for CCK8s and CCKBR agonist (3r1) treatment in juvenile after Long-term Monocular Deprivation (LTMD) (P21-P42);

FIG. 1C shows an embodiment of the raw spike traces after LTMD in juvenile followed by saline (infusion/i.p) and CCK8s/CCKBR agonist (3r1) between deprived eye and non-deprived eye:

FIG. 1D shows an embodiment of Contralateral Bias Index (CBI) of individual mice among the different groups:

FIG. 1E shows an embodiment of the histogram of the visual cortex cells with contralateral bias index (CBI) in adult (postnatal day 60 (P60)) with no Monocular Deprivation (MD);

FIG. 1F shows an embodiment of the histogram of the visual cortex cells with CBI after LTMD in juvenile followed by saline (infusion);

FIG. 1G shows an embodiment of the histogram of the visual cortex cells with CBI after LTMD in juvenile followed by CCK8s treatment for 15 days:

FIG. 1H shows an embodiment of the histogram of the visual cortex cells with CBI after LTMD in juvenile followed by saline (i.p):

FIG. 1I shows an embodiment of the histogram of the visual cortex cells with CBI after LTMD in juvenile followed by CCKBR agonist (3r1) treatment for seven days:

FIG. 1J shows an embodiment of the histogram of the visual cortex cells with CBI after LTMD in juvenile followed by CCKBR agonist (3r1) treatment for fourteen days:

FIGS. 2A and 2B show embodiments of the visual behavioral experimental design for CCK8s and CCKBR agonist (3r1) treatment in juvenile after LTMD;

FIG. 2C shows embodiments of visual acuity of wild type (WT) adult (P60) mice with no MD;

FIG. 2D shows embodiments of visual acuity of LTMD mice followed by saline (infusion)/CCK8s treatment:

FIG. 2E shows embodiments of visual acuity of LTMD mice followed by saline (i.p)/CCKBR agonist (3r1) treatment:

FIG. 2F shows an embodiment of percentage accuracy achieved by each group before and after training; and

FIG. 2G shows an embodiment of the Mean±SEM cycle/degree (cpd) of each group during the testing after treatment.

The figures herein are for illustrative purposes only and are not necessarily drawn to scale.

### DESCRIPTION OF THE PREFERRED EMBODIMENTS

Unless otherwise specifically provided, all tests herein are conducted at standard conditions which include a room and testing temperature of 25° C., sea level (1 atm.) pressure, pH 7, and all measurements are made in metric units. Furthermore, all percentages, ratios, etc. herein are by weight, unless specifically indicated otherwise. It is understood that unless otherwise specifically noted, the materials compounds, chemicals, etc. described herein are typically commodity items and/or industry-standard items available from a variety of suppliers worldwide.

An embodiment of the present invention relates to a method for treating or preventing amblyopia by administrating a therapeutically effective amount of cholecystokinin receptor B (CCKBR) agonist in a subject in need thereof.

Without intending to be limited by theory, it is believed that amblyopia disables a normal development of visual acuity in the anatomically intact eye. During visual development, the better-seeing eye receives a larger share of the available synapses in the visual cortex than the poorer-seeing eye. More synapses in the cortex may be controlled by the better-seeing eye than is required for optimal vision. Because the visual cortex has a limited number of synapses, the impaired eye may have fewer synapses than is required for normal vision. If this scenario is not corrected before the critical period (CP) of visual development, these synaptic connections may become permanent, and neither the underlying disadvantage of the weaker eye nor patching therapy will restore normal visual acuity to the eye. Treatment may be achievable if this problem is detected during the CP of visual development. However, it is believed that occlusive therapy becomes less effective as the patient ages, with adults receiving little or no benefit.

Without intending to be limited by theory, it is believed that CP is the key time when sensory experience is necessary for normal circuit development. CP is also crucial for the experience-dependent refinement of the cortical circuits. All the neuronal connections are malleable during this period. Towards the end of the CP, the experience-dependent plasticity of the neural circuitry ceases to occur. Abnormal visual input to one eye during infancy may result in permanent loss of visual acuity, or amblyopia, if not corrected during childhood. The period of P21-P38 is known as the critical period of ocular dominance in mice.

This invention fulfills the demand for a treatment to improve vision in an amblyopic eye. In particular, the invention surprisingly provides a new method to cause a long-lasting but reversible blockage of optic nerve transmission in the dominant eye, allowing complete or considerable visual recovery in the amblyopic eye. The methods described herein are especially well suited to treat adults who have not responded to typical child-centered therapies. The inventors has surprisingly found that CCKBR agonist may facilitate the recovery of lost visual plasticity, especially in adults.

In an embodiment herein, the CCKBR agonist is administrated at a dosage of from about 10 nM/kg to about 4.18 uM/kg. In some embodiments, the CCKBR agonist can be effective at even a low dosage of about 0.0122 mg/kg, which will minimize side effects.

In an embodiment herein, the CCKBR agonist is administrated at a frequency of about 4 times a week or 7 times two weeks, or alternatively, it may be administrated once every other day for about one to two weeks.

In an embodiment herein, the subject is treated with the method provided herein for a period of at least two weeks, for example, from about 4 weeks to about 6 months. In some embodiments, the subject is treated for a period of about 4 weeks, about 5 weeks, about 6 weeks, about 1.5 months, about 2 months, about 3 months, about 4 months, about 5 months, or about 6 months.

In an embodiment herein, the CCKBR agonist is administrated via a route selected from the group of intraperitoneal administration, intracerebroventricular administration, oral administration, subcutaneous injection, and a combination thereof. In certain embodiments, intraperitoneal administration is selected because it may have maximum bioavailability and the agonist directly enters the blood circulation. Without intending to be limited by theory, it is believed that CCKBR agonist can cross the blood brain barrier (BBB) and it can stay for longer time in the circulation due to its long half-life.

In an embodiment herein, the CCKBR agonist is selected from the group of CCK8s, CCK4, 3r1 (having a structure of Ac-Trp-Nle-Asp-Phe(3-Br)—NH₂), and a combination thereof. CCK and its metabolites (CCK8s) are natural agonists. Whereas, 3r1 is a synthetic molecule, which may have properties better than CCK. Specifically, without intending to be limited by theory, it is believed that 3r1 can cross the blood brain barrier, and 3r1 has a half-life longer than CCK8s agonist, hence it may stay for longer time before being degraded and eliminated from the body. Without being limited by theory it is believed that the mechanism of action of CCK and its agonist is via CCKB receptors.

In an embodiment herein, the subject being treated with the methods herein is selected from the group of an infant, a child, an adult, and a combination thereof. In some embodiments, the method provided herein shows significant superiority in treating adults because it provides a new chance of developing normal brain-eye connections that didn't happen at early ages.

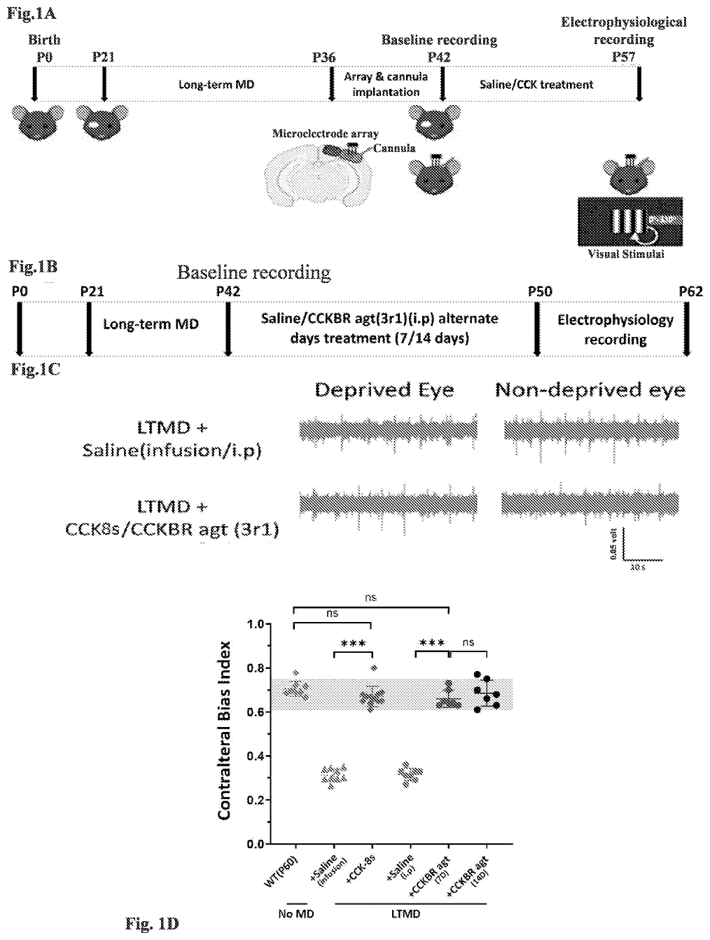

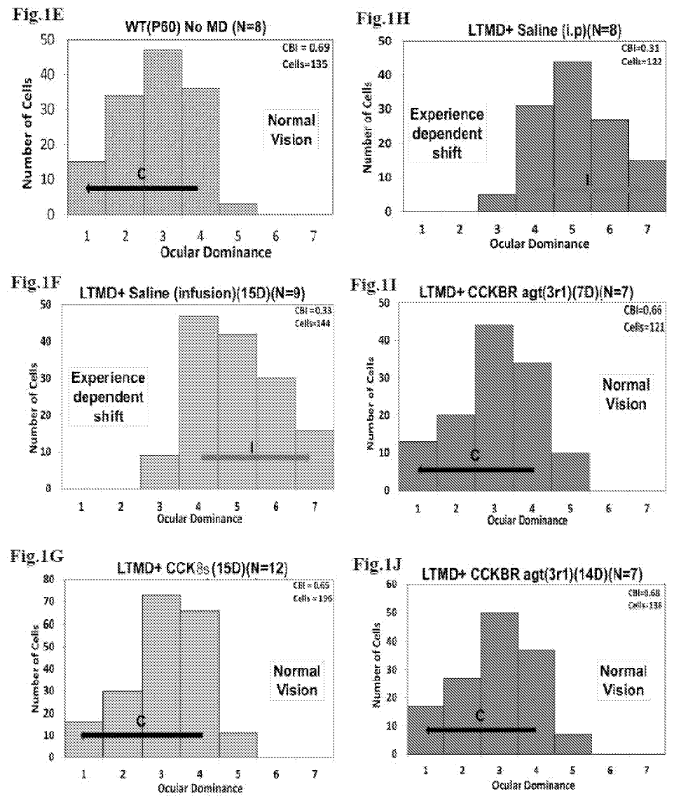

The present inventions prove that CCKBR agonist (such as 3r1) may open the critical period of plasticity in adulthood. As illustrated in the embodiment of FIG. 1, CCK8s and CCKBR agonist (3r1) treatment reverses the effect of LTMD in juvenile C57BL/6 mice model of amblyopia. FIG. 1D shows CBI of individual mice among the different groups. As shown in FIG. 1D, the gray box indicates the typical range of CBI values for the mice. Solid black line represents the average CBI score for each group. A significant difference in CBI is observed between the juvenile LTMD following the saline (infusion) and CCK8s (15 days) treatment group (*** P<0.001) and between the juvenile LTMD following the saline (i.p) and CCKBR agonist (3r1) (7 days) treatment group (*** P<0.001). No significant difference in CBI is observed between the adult (P60) with no MD and juvenile LTMD following CCK8s (15 days)/CCKBR agonist (3r1) (7 days) treatment group (n.s., P>0.05). No Significant difference is observed in CBI between CCKBR agonist (3r1) 7 days and 14 days' treatment group (n.s., P>0.05).

FIGS. 1E-1J show the histogram of the visual cortex cells with contralateral bias index (CBI). In adult (P60) with no MD (FIG. 1E), a histogram shows the V1 cells with strong contralateral eye bias. After LTMD in juvenile followed by saline (FIG. 1F), histogram shows the shift in the eye response, V1 cells with strong ipsilateral eye bias. After LTMD in juvenile followed by CCK8s treatment for 15 days (FIG. 1G), histogram shows the shift in the eye response, V1 cells with strong contralateral eye bias. After LTMD in juvenile followed by saline (FIG. 1H), histogram shows the shift in the eye response, V1 cells with strong ipsilateral eye bias. After LTMD in juvenile followed by CCKBR agonist (3r1) treatment for seven days (FIG. 1I), histogram shows the shift in the eye response, V1 cells with strong contralateral eye bias. After LTMD in juvenile followed by CCKBR agonist (3r1) treatment for fourteen days (FIG. 1J), histogram shows the shift in the eye response, V1 cells with strong contralateral eye bias. Error bars in FIG. 1D indicate the standard error of the mean (SEM).

An embodiment of the present invention relates to a method for regaining or improving visual acuity by administrating a therapeutically effective amount of CCKBR agonist in a subject in need thereof. The above described parameters of the method for treating or preventing amblyopia also apply to this embodiment of the invention.

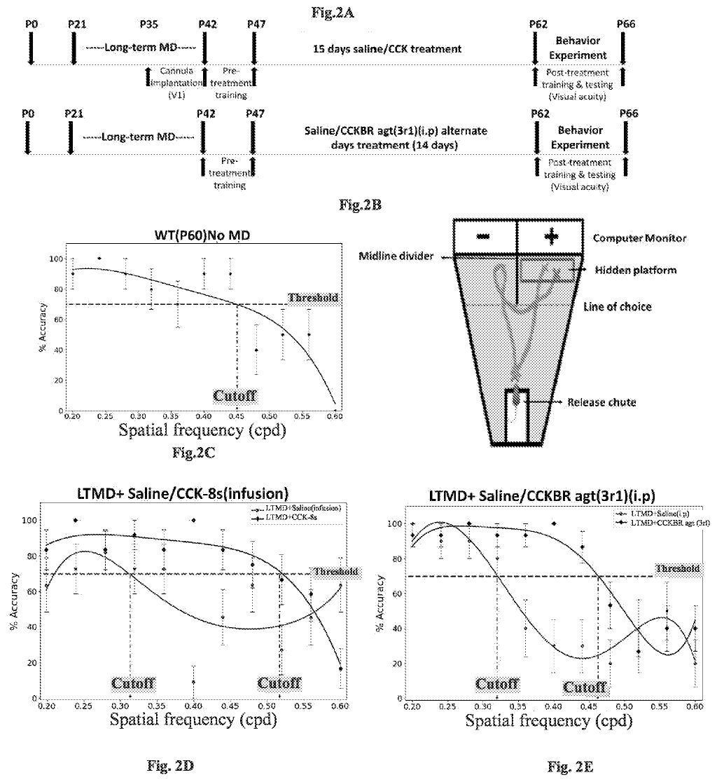

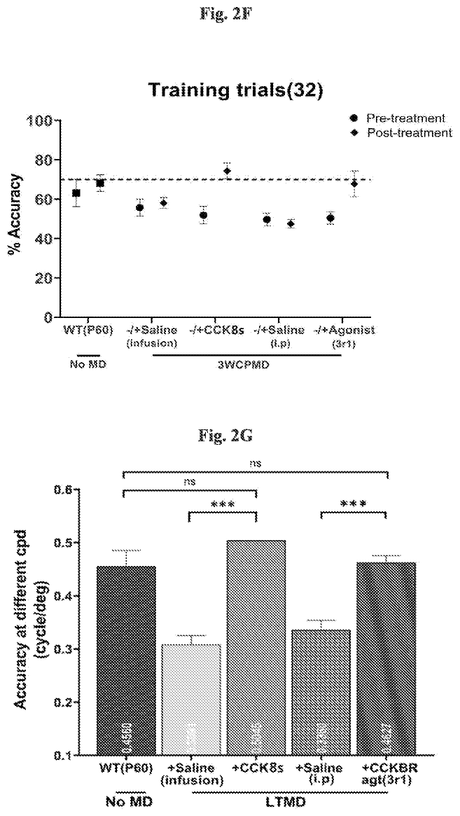

Amblyopia patients usually have poor visual acuity. It is believed that the method according to the invention can reverse the lost visual acuity in amblyopia patients. FIGS. 2A-2G illustrate the assessment of visual acuity using visual water maze. Dotted line represents the 70% accuracy as cutoff. As shown in FIGS. 2C-2E, WT adult (P60) mice with no MD show cutoff at cpd 0.45 achieved during testing by all the animals (12 animals). After LTMD in juvenile followed by saline (infusion)/CCK8s treatment, the mice show cutoff at cpd 0.30 and 0.52 respectively achieved during testing by all the animals (11 animals/12 animals respectively). After LTMD in juvenile followed by saline (i.p)/CCKBR agonist (3r1) treatment, the mice show cutoff at cpd 0.33 and 0.46 respectively achieved during testing by all the animals (11 animals/15 animals respectively). FIG. 2F shows the percentage accuracy achieved by all animals before and after training. The performance of the animals during training in WT adult (P60) with no MD, before and after remained unchanged. After LTMD in juvenile, the performance of animals during training before and after saline (infusion) treatment remained unchanged and the performance of animals during training before and after CCK8s treatment show the difference. After CCK8s treatment the performance reaches beyond the cutoff. After LTMD in juvenile, the performance of animals during training before and after saline (i.p) treatment remained unchanged and the performance of animals during training before and after CCK-agonist (3r1) treatment show the difference. After CCKBR agonist (3r1) treatment the performance reaches beyond the cutoff. As shown in FIG. 2G, the visual acuity is observed in juvenile after LTMD. A significant difference is observed between saline (infusion) and CCK8s treated animals (P<0.001) and in between saline (i.p) and CCKBR agonist (3r1) (P<0.001). No significant difference in visual acuity observed in adult (P60), no MD and juvenile LTMD following CCK8s and CCKBR agonist (3r1) treated animals (P>0.05). Error bars in FIG. 2G indicate the standard error of the mean (SEM).

An embodiment of the present invention relates to a use of a CCKBR agonist in the manufacture of a medicament for treating or preventing amblyopia in a subject in need thereof. The above described parameters of the method for treating or preventing amblyopia also apply to this embodiment of the invention.

In an embodiment herein, the medicament may be in a form of solution or powder.

### EXAMPLES

### Example 1

### Electrophysiology Results: CCK BR Agonist Opens the Critical Period of Plasticity in Adulthood

### Surgical Procedure

The mice are anesthetized through the application of a K-X combination during the surgical preparation. The body temperatures of the mice are maintained at 37° C. by use of a homeostatically controlled heating pad during the surgery. Following the induction of anesthesia, each mouse is mounted on a stereotaxic instrument (RWD Life Science, Shenzhen, China) and an incision is made along the midline of the head. The stereotaxic instrument is adjusted to place the bregma and lambda points of the skull on a flat skull surface (anteroposterior: −3.2 mm to −4.0 mm; mediolateral: 3.0-3.8 mm, relative to bregma). A piece of the scalp over the V1 is removed to expose the cortex, and a bone screw is fixed in the frontal bone as the reference electrode. The exposed cortical surface is then covered with extracellular saline (in mM: 125 NaCl, 5 KCl, 10 glucose, 10 4-(2-hydroxyethyl)-1-piperazineethanesulphonicacid [HEPES], and 2 CaCl₂; pH adjusted to 7.4) to prevent drying of the surface. The electrode is lowered into the brain to an appropriate depth (V1≤1 mm) and is allowed to settle for 30 min before recording is begun. In each mouse, 3-4 separate penetrations are spaced evenly at least 200 μm apart across the binocular region of V1. The eyes are kept moist with ophthalmic lubricant ointment until recording to prevent drying while enabling clear optical transmission. At the end of the recording, the mice are euthanized by injection of an overdose of Dorminal (300 mg/kg).

### Spike Recording

Visual stimuli of sinusoidal drifting gratings with increasing orientation angles(0-330°) and 0.4 cpd are presented in random order to alternate eyes. The spike recordings are performed with the use of a polyimide-coated platinum/iridium (70:30) ribbon microelectrode array (Clunbury Scientific LLC, USA). The tip resistances are 30-50 kΩ. The A-M Systems 3600 (A-M Systems, USA) and CED Micro 1401-3 (Cambridge Electronic Design, UK) instruments are used as the amplifier and data acquisition system, respectively. The signals are sampled at 25 kHz and bandpass-filtered at 300 Hz-3 KHz for spike, amplified, and fed to spike2 software (Cambridge Electronic Design, UK). Offline analysis is performed using MATLAB (R2020b, Math Works, USA). To begin, the raw data are transformed into a MATLAB-compatible format. Spike sorting is used to distinguish various units collected at the same time in the spike data. Wave clus, a MATLAB tool, is used to categorize the units based on the characteristics of their waveforms.

The neuronal response in the Visual cortex (V1) is recorded extracellularly, while the same visual stimulus with varied orientation is presented to either of the eyes alternatively, and the relative strength of the response is determined.

To quantify the deprivation effect, the inventors calculate the contralateral bias index (CBI) from spikes (response from the single neuron) using the following formula:

$$CBI = \frac{( {N}_{1} - {N}_{7} ) + \frac{2}{3} ( {N}_{2} - {N}_{6} ) + \frac{1}{3} ( {N}_{3} - {N}_{5} ) + {N}_{T}}{2 \cdot {N}_{T}}$$

where N_{T} is the total number of units and N_{X} is the number of units with OD scores equal to x (x=1, 2, 3, 5, 6, 7). For the sum of the number of cells in each category is used to calculate the CBI for each animal.

A CBI of 1 indicates that the hemisphere responds only to the inputs from the contralateral eye, whereas a CBI of 0 indicates that the hemisphere responds only to the inputs from the ipsilateral eye. The test results of this example are shown in FIG. 1D.

Three groups of experiment are performed in this example. In the first experiment, the eye preference of the single neurons (ocular dominance score) is calculated in, wild type (WT) mice at postnatal day (P60). The results show that WT (P60) mice with no MD have higher CBI (CBI=0.69, 8 mice) indicating neuroplasticity does not occur in adulthood. The eye response is dominated by the contralateral eye (FIG. 1E).

FIGS. 1A and 1B, show the timeline of the experimental setup, i.e. LTMD, surgery, dosing, and in-vivo electrophysiology used in the second and third experiments. In the second experiments, in juvenile after LTMD, saline or CCK8s (Cholecystokinin octapeptide (Tocris Bioscience, UK): 1 ul each day (10 ng/ul), Cholecystokinin octapeptide (Tocris Bioscience, UK)) is infused (treatment) for 15 days (from P42 to P57) in V1. In juvenile after LTMD, saline treatment for 15 days does not affect eye dominance (CBI=0.33, 9 mice) (FIG. 1F). The ipsilateral eye is dominated. Contrarily, in the group treated with CCK8s (infusion in V1), the ocular dominance shifts from the ipsilateral eye towards the contralateral eye (CBI=0.65, 12 animals) (FIG. 1G). It is surprisingly found that CCK8s reverses the eye response effect of juvenile LTMD.

In the third experiments, in juvenile after LTMD, saline treatment (i.p) for 15 days does not affect eye dominance (CBI=0.31, 8 animals). In juvenile after LTMD, 3r1 (a type of CCKBR agonist, Guangzhou Institute of Biomedicine and Health, affiliated with the Chinese Academy of Sciences, Guangzhou, China) (4.18 uM) is administered intraperitoneally (i.p). CCKBR agonist (3r1)(i.p) is administered via two different drug dose frequencies, i.e., a) 4 doses on alternate days for 7 days; and b) 8 doses on alternate days for 14 days. One dose refers to 0.0122890 mg/kg/i.p/mice. After LTMD, saline administration (i.p) for 14 days does not affect eye dominance (CBI=0.31, 8 animals) (FIG. 1H). The ipsilateral eye is dominated. Contrarily, CCKBR agonist, i.e., 3r1 (i.p) reverses the ocular dominance so that it shifts from the ipsilateral eye towards the contralateral eye. The shift is observed in both 7 days CCKBR agonist (3r1)(i.p) treatment (CBI=0.66, 7 animals) (FIG. 1I) and 14 days CCK BR agonist (3r1)(i.p) treatment (CBI=0.68, 7 animals) (FIG. 1J). The results show CCKBR agonist (3r1) leads to opening of critical period plasticity in adulthood.

### Example 2

### Behavioural Assay: CCKBR Agonist Treatment Restores Visual Acuity in the Amblyopic Eye

Visual water behavioral test is performed to show visual acuity improvement following long-term monocular deprivation after administration of CCK8s and CCKBR agonist (3r1). Each animal learns quickly to associate swimming to the escape platform placed below the screen displaying grating stimuli during pre-training phase and post-training phase. Animals of all groups learn serendipitously during training to swim to the end of the divider, and then look at each screen several times before making their choice.

FIGS. 2A and 2B show the behavioural experimental design of this example: (CCK8s) infusion in V1 and CCKBR agonist (3r1)(i.p). The testing criterion in the training phase (from P42 to P47) is reached for all mice within four sessions (32 trials). Average performance above 70% accuracy is maintained for all mice of different groups (FIG. 2F). The visual acuity of the groups is illustrated in FIGS. 2C-2E. The visual acuity of mice with different treatments is listed in the table below:

| Mice Treatments | NO. of Mice | Visual acuity | |
|---|---|---|---|
| the WT adult (P60) | 12 mice | 0.44 | c/d |
| Long-term MD (P21-P42) + | 11 mice | 0.3 | c/d |
| saline (infusion) | | | |
| Long-term MD (P21-P42) + | 12 mice | 0.5 | c/d |
| CCK (infusion) | | | |
| Long-term MD (P21-P42) + | 11 mice | 0.33 | c/d |
| saline (i.p) | | | |
| Long-term MD (P21-P42) + | 15 mice | 0.46 | c/d |
| CCKBR agonist (3r1) (i.p) | | | |

Post hoc analysis is conducted. The results (FIG. 2G) show that, no significant difference in the accuracy at different cpd is observed in WT adult (P60) with no MD vs. in juvenile after LTMD+CCK treated animals (0.44±0.02 c/d vs 0.5±0.01, P>0.05). In contrast, the significant difference in the accuracy at different cpd is observed in juvenile after LTMD+CCK treated animals vs. in juvenile after LTMD+saline-treated animals (0.5±0.01 vs 0.309±0.01, P<0.001). No significant difference in the accuracy at different cpd is observed in WT adult (P60) with no MD vs. in juvenile after LTMD+CCKBR agonist (3r1) treated animals (0.44±0.02 c/d vs 0.46±0.02, P>0.05). In contrast, the significant difference in the accuracy at different cpd is observed in juvenile after LTMD+CCKBR agonist (3r1) animals vs. in juvenile after LTMD+saline (i.p)-treated animals and (0.46±0.02 vs 0.33±0.01, P<0.001).

The above finding shows that the CCK8s infusion and CCKBR agonist (3r1)(i.p) treatment in animals post LTMD, restores the visual acuity in all animals same as WT adults. The behavioral experiment results comply with the electrophysiology results.

It should be understood that the above only illustrates and describes examples whereby the present invention may be carried out, and that modifications and/or alterations may be made thereto without departing from the spirit of the invention.

It should also be understood that certain features of the invention, which are, for clarity, described in the context of separate embodiments, may also be provided in combination in a single embodiment. Conversely, various features of the invention which are, for brevity, described in the context of a single embodiment, may also be provided separately, or in any suitable sub combination.

All references specifically cited herein are hereby incorporated by reference in their entireties. However, the citation or incorporation of such a reference is not necessarily an admission as to its appropriateness, citability, and/or availability as prior art to/against the present invention.

1. A method for treating or preventing amblyopia by administrating a therapeutically effective amount of cholecystokinin receptor B (CCKBR) agonist in a subject in need thereof.

2. The method of claim 1, wherein the CCKBR agonist is administrated at a dosage of from about 10 nM/kg to about 4.18 uM/kg.

3. The method of claim 2, wherein the CCKBR agonist is administrated at a frequency of about 4 times a week.

4. The method of claim 1, wherein the subject is treated for a period of from about 4 weeks to about 6 months.

5. The method of claim 1, wherein the CCKBR agonist is administrated via intraperitoneal administration.

6. The method of claim 1, wherein the CCKBR agonist is selected from the group consisting of CCK8s, CCK4, 3r1, and a combination thereof.

7. The method of claim 1, wherein the subject is human.

8. The method of claim 1, wherein the subject is selected from the group consisting of an infant, a child, an adult, and a combination thereof.

9. A method of regaining or improving visual acuity by administrating a therapeutically effective amount of CCKBR agonist in a subject in need thereof.

10. The method of claim 9, wherein the CCKBR agonist is administrated at a dosage of from about 10 nM/kg to about 4.18 uM/kg.

11. The method of claim 10, wherein the CCKBR agonist is administrated at a frequency of about 4 times a week.

12. The method of claim 9, wherein the subject is treated for a period of about 4 weeks to about 6 months.

13. The method of claim 9, wherein the CCKBR agonist is administrated via intraperitoneal administration.

14. The method of claim 9, wherein the CCKBR agonist is selected from the group consisting of CCK8s, CCK4, 3r1, and a combination thereof.

15. The method of claim 9, wherein the CCKBR agonist leads to opening of critical period of neuroplasticity in visual cortex.

16. The method of claim 9, wherein the subject is human.

17. Use of a CCKBR agonist in the manufacture of a medicament for treating or preventing amblyopia in a subject in need thereof.

18. The use of claim 17, wherein the medicament is in a form of solution.

19. The use of claim 17, wherein the CCKBR agonist is selected from the group consisting of CCK8s, CCK4, 3r1, and a combination thereof.

20. The use of claim 17, wherein the subject is selected from the group consisting of an infant, a child, an adult, and a combination thereof.
